(19) United States
(12) Patent Application Publication
(10) Pub. No.: US 2021/0146161 A1
(43) Pub. Date: May 20, 2021

(54) SYSTEMS AND METHODS FOR PROVIDING AUTOMATED QUALITY ASSURANCE IN RADIATION ONCOLOGY

(71) Applicant: The Moses H. Cone Memorial Hospital Operating Corporation, Greensboro, NC (US)

(72) Inventors: Benjamin Jeremiah Sintay, Summerfield, NC (US); David Brian Wiant, Kernersville, NC (US)

(73) Assignee: The Moses H. Cone Memorial Hospital Operating Corporation, Greensboro, NC (US)

(21) Appl. No.: 17/098,784

(22) Filed: Nov. 16, 2020

Related U.S. Application Data
(60) Provisional application No. 62/936,019, filed on Nov. 15, 2019.

(51) Int. Cl. A61N 5/10; G16H 40/20; G16H 20/40; G16H 70/20; G16H 40/67; G16H 40/40; G16H 15/00; G16H 20/10; G16H 30/20; G16H 30/40

(52) U.S. Cl. CPC A61N 5/1075; A61N 5/1067; A61N 5/1069; A61N 5/103; G16H 40/20; G16H 20/40; A61N 2005/1074; G16H 40/67; G16H 40/40; G16H 15/00; G16H 20/10; G16H 30/20; G16H 30/40; G16H 70/20

(57) ABSTRACT

Systems and methods for an oncology radiation therapy treatment review are disclosed. The systems include a radiation therapy delivery system, a processor, and a non-transitory memory containing computer-readable instructions. The instructions are executed by the processor to receive information relating to a subject undergoing radiation therapy treatment in accordance with a treatment plan, identify at least one clinical requirement associated with the treatment plan, and analyze the received information and the at least one clinical requirement for identifying an action, the action comprising at least one of the following: configuring a user interface, performing a corrective action, storing results of the analyzing to a database, outputting an alert, or disabling treatment, or a document related action.

### CROSS-REFERENCE TO RELATED APPLICATIONS

This application claims priority to provisional application Ser. No. 62/936,019 filed on Nov. 15, 2019, the entirety of which is hereby incorporated by reference.

### BACKGROUND

Subjects receiving any form of healthcare (including radiation therapy) must have detailed records of care, commonly referred to as a medical record. In recent years, healthcare service providers have been making the transition from manual paper-based medical records to an electronic format in a hospital medical record system and/or other disease-specific information system. Electronic medical records substantially increase the efficiency of healthcare providers and institutions. Electronic medical records also substantially reduce risks associated with high volumes of subject data and potential liabilities arising out of clerical errors or misinformation. The electronic format enhances communication between various providers and within institutions. As electronic clinical documentation continues to become increasingly prevalent, the variety of applications, electronic forms, electronic charts, and user interfaces, as well as the corresponding versatility of this format, continue to expand.

In general, a radiation therapy (RT) for the treatment of cancer is a method that radiates an optimal radiation dose onto cancer tissue while radiating a minimum radiation dose onto normal tissue around the cancer tissue, thereby improving the effects of cancer treatment without damaging the normal tissue. The delivery of radiation therapy for the treatment of cancer typically is a complicated process that requires both clinical and technical expertise in order to generate treatment plans that are safe and effective for the treatment of cancer. As such, RT treatment plan quality assurance (QA) process relies on the vigilance of the multi-disciplinary team to review and assimilate relatively complex data from different sources. A multi-disciplinary RT team comprising radiation therapists, physicists and oncologists typically reviews each proposed treatment plan for clinical and technical merit. This review includes assessing safety (e.g., that the proposed plan does not exceed any normal tissue dose tolerances), deliverability (e.g., the dose calculated in the proposed treatment plan can be reproduced on the treatment unit), consistency in the transfer of data between databases (e.g., the parameters defining the proposed plan are the same parameters to actually treat the specific subject) and overall quality (e.g., the proposed plan is consistent with other plans for the given site and technique in terms of the dose prescription, the dose distribution, target coverage etc.). Currently, this process is largely manual and complex, as there may be numerous parameters that require human expert review. This has led to an interest in automated QA methods in order to reduce the reliance on human vigilance.

The systems and methods of this disclosure address the issues discussed above.

The information included in this Background section of the specification, including any references cited herein and any description or discussion thereof, is included for technical reference purposes only and is not to be regarded subject matter by which the scope of the invention as defined in the claims is to be bound.

### BRIEF DESCRIPTION OF THE DRAWINGS

FIG. 1 illustrates a schematic of an example system for providing automated quality assurance in radiation oncology.

FIG. 2 is a flowchart illustrating an example method for performing automated quality assurance in radiation oncology.

FIG. 3 illustrates an example user interface generated in accordance with this disclosure.

FIG. 4 illustrates an example of internal hardware that may be included in any of the electronic components of the system.

### SUMMARY

In some scenarios, disclosed is systems and methods for an oncology radiation therapy treatment review are disclosed. The systems may include a radiation therapy delivery system, a processor, and a non-transitory memory containing computer-readable instructions. The instructions may executed by the processor to perform the disclosed methods. The system may receive information relating to a subject undergoing radiation therapy treatment in accordance with a treatment plan, identify at least one clinical requirement associated with the treatment plan, and analyze the received information and the at least one clinical requirement for identifying an action, the action comprising at least one of the following: configuring a user interface, performing a corrective action, storing results of the analyzing to a database, outputting an alert, or disabling treatment, or a document related action. Optionally, the system may also control the radiation therapy delivery system to deliver the radiation therapy treatment. Additionally and/or alternatively, the system may also automatically identify the treatment plan.

In various implementations, the system may analyze a plurality of technical parameters of the treatment plan and using the analysis to control the radiation therapy delivery system to deliver the radiation therapy treatment. Examples of the technical parameters can include, without limitation, gantry angle, collimator angle, beam isocenter position, collimator jaw setting, beam placement, beam shaping, dose distributions, dose volume statistics, and beam strength. Examples of the clinical requirement can include, without limitation, amount of non-target tissue that may be irradiated, minimum amount of coverage of the target area, prioritization of areas to irradiate, and prioritization of areas to avoid.

In certain implementations, the system may also include a display coupled to the processor for displaying the user interface, and an input device coupled to the processor for receiving a user input. Optionally, the action may include configuring the user interface to display the received information and results of the analyzing in a hierarchical manner, and displaying it on the display.

Optionally, analyzing the received information and the at least one clinical requirement for identifying the action may include identifying one or more errors in documentation required for the treatment plan, identifying a corrective action corresponding to the one or more errors, and performing the corrective action. Optionally, the system may also automatically identify one or more errors in a prescription or an order associated with the treatment plan.

In certain implementations the system may analyze the received information and the at least one clinical requirement for identifying the action in order to disable the treatment delivery system until one or more discrepancies identified in the treatment plan have been resolved by the system.

Additionally and/or alternatively, examples of the action may include, without limitation, initiating a manual chart review or performing an automatic chart review.

The system, in certain implementations, may analyze the received information and the at least one clinical requirement for identifying the action to determine compliance with one or more quality assurance rules relating to the treatment plan and/or to determine one or more errors associated with a plurality of images corresponding to the treatment plan.

### Specification

In the following detailed description, reference is made to the accompanying drawings, which form a part hereof. In the drawings, similar symbols typically identify similar components, unless context dictates otherwise. The illustrative examples described in the detailed description, drawings, and claims are not meant to be limiting. Other examples may be utilized, and other changes may be made, without departing from the spirit or scope of the subject matter presented herein. It will be readily understood that the aspects of the present disclosure, as generally described herein and illustrated in the Figures, can be arranged, substituted, combined, separated, and designed in a wide variety of different configurations, all of which are implicitly contemplated herein.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to.” Definitions for additional terms that are relevant to this document are included at the end of this Detailed Description.

Terminology that is relevant to the disclosure provided above includes:

The term “machine learning model” or a “model” refers to a set of algorithmic routines and parameters that can predict an output(s) of a real-world process (e.g., identification of anomalies in a treatment plant and/or treatment plan delivery, a suitable recommendation based on a user search query, etc.) based on a set of input features, without being explicitly programmed. A structure of the software routines (e.g., number of subroutines and relation between them) and/or the values of the parameters can be determined in a training process, which can use actual results of the real-world process that is being modeled. Such systems or models are understood to be necessarily rooted in computer technology, and in fact, cannot be implemented or even exist in the absence of computing technology. While machine learning systems utilize various types of statistical analyses, machine learning systems are distinguished from statistical analyses by virtue of the ability to learn without explicit programming and being rooted in computer technology.

A typical machine learning pipeline may include building a machine learning model from a sample dataset (referred to as a “training set”), evaluating the model against one or more additional sample datasets (referred to as a “validation set” and/or a “test set”) to decide whether to keep the model and to benchmark how good the model is, and using the model in “production” to make predictions or decisions against live input data captured by an application service.

An “electronic device” or a “computing device” refers to a device that includes a processor and memory. Each device may have its own processor and/or memory, or the processor and/or memory may be shared with other devices as in a virtual machine or container arrangement. The memory will contain or receive programming instructions that, when executed by the processor, cause the electronic device to perform one or more operations according to the programming instructions.

The terms “memory,” “memory device,” “data store,” “data storage facility” and the like each refer to a non-transitory device on which computer-readable data, programming instructions or both are stored. Except where specifically stated otherwise, the terms “memory,” “memory device,” “data store,” “data storage facility” and the like are intended to include single device embodiments, embodiments in which multiple memory devices together or collectively store a set of data or instructions, as well as individual sectors within such devices.

The terms “processor” and “processing device” refer to a hardware component of an electronic device that is configured to execute programming instructions. Except where specifically stated otherwise, the singular term “processor” or “processing device” is intended to include both single-processing device embodiments and embodiments in which multiple processing devices together or collectively perform a process.

In this document, the terms “communication link” and “communication path” mean a wired or wireless path via which a first device sends communication signals to and/or receives communication signals from one or more other devices. Devices are “communicatively connected” or “communicatively coupled” if the devices are able to send and/or receive data via a communication link. “Electronic communication” refers to the transmission of data via one or more signals between two or more electronic devices, whether through a wired or wireless network, and whether directly or indirectly via one or more intermediary devices.

In this document, when relative terms of order such as “first” and “second” are used to modify a noun, such use is simply intended to distinguish one item from another, and is not intended to require a sequential order unless specifically stated.

In addition, terms of relative position such as “vertical” and “horizontal”, or “front” and “rear”, when used, are intended to be relative to each other and need not be absolute, and only refer to one possible position of the device associated with those terms depending on the device's orientation.

As used herein the term “electronic medical records (EMR)” include, for example and without limitation, subject history, physician notes, diagnoses, prescriptions, physician orders, medical images, operative reports, test results, and treatment-related information. Treatment-related information may include technical parameters relevant to a specific therapy. The records may be displayed with other information about a subject in a user reviewable format referred to as a “chart”.

As used herein, the term “subject” refers to any animal (e.g., a mammal), including, but not limited to, humans, non-human primates, rodents, and the like, which is to be the recipient of a particular diagnostic test or treatment. Typically, the terms “subject” and “patient” are used interchangeably herein in reference to a human subject.

The term “clinical requirement” may be used to refer to any treatment-specific or outcome-specific requirement that may be specified by a user (e.g., a clinician or oncologist) and/or automatically. Typical clinical requirements for radiotherapy planning may include, for example: amount of non-target tissue that may be irradiated, minimum amount of coverage of the target area, and prioritization of which areas to irradiate/avoid. A single clinical requirement may require multiple technical parameters in order to be satisfied. Thus, change in a single clinical requirement may require adjusting multiple technical parameters. Clinical requirements are often inter-related, overlapping or even contradictory, and may require proper prioritization and complex calculations of the technical requirements in order to achieve a clinically acceptable treatment plan.

The term “technical parameter” may be used to refer to any parameter that controls the technical operation of the treatment system. Typical technical parameters may include, for example: gantry angle, collimator angle, beam isocenter position, collimator jaw setting, beam placement, beam shaping, dose distributions, dose volume statistics, and beam strength (i.e., dosage). Examples are discussed in greater detail below. A single technical parameter may affect multiple clinical outcomes. Thus, change in a single technical parameter may affect whether multiple clinical requirements are satisfied. The relationship between each technical parameter and each clinical outcome (and the degree to which each clinical requirement is satisfied) may not be intuitive or easily understood. In conventional treatment planning, technical parameters may be manually selected based on the personal experience, intuition and knowledge of the clinician or radiation therapy treatment planner (dosimetrist), usually involving a trial-and-error approach, and thus may not be reproducible, reliable or easy to determine. In the presently disclosed systems and methods, a technical parameter may be automatically determined based on one or more empirical rules/relationships, or may be automatically calculated using an optimization parameters and/or machine learning models.

Radiation oncology is the discipline within healthcare that uses targeted high-energy radiation to treat malignant, and occasionally, benign diseases. The process of assessing and preparing a subject for treatment requires numerous points of documentation. Preparation for treatment includes the creation of a treatment plan, typically customized for an individual subject's anatomy and needs. Treatment plans are complex and frequently consist of medical images, anatomic contours (i.e., delineation of specific anatomy or cancerous tissues being targeted), treatment beams, and computed radiation dose held within a complex computer system. Treatment plans, their resulting data, and reports are carefully reviewed prior to treatment by multiple staff members including physicians, qualified medical physicists, medical dosimetrists, and radiation therapists. Specifically, Radiation oncology treatment delivery is a complex process that involves multiple procedures, technologies, and health care providers working together.

The high degree of complexity leaves the execution of this process open to a number of different errors. To help mitigate these errors many safeguards and layers of quality assurance are practiced. One key area of quality assurance is management of care by a certified medical physicist. The American Association of Physicists in Medicine (AAPM) recommends a thorough initial, weekly, and end of treatment review of each subject's medical records by a qualified medical physicist. For example, one of the most important, complex and time-consuming of these checks is the pre-treatment physics chart review (TPCR). This check is performed prior to the start of treatment to verify the safety, integrity and consistency of all aspects of the treatment chart from the documentation to the integrity of the CT used for planning, the quality and safety of the planned treatment and the correctness of the transferred plan in the electronic medical record system. Treatment cannot start until this review is complete and the RT chart has been determined to be safe and free of errors. If the treatment plan contains errors and is therefore not approved for treatment, a fail safe interlock on the treatment machine prevents the radiation from being delivered to the subject. Additional weekly review is required by physicians in the weekly management of subjects under treatment. In some practices other staff in radiation oncology, including radiation therapists and medical dosimetrists, initially and periodically review charts. Chart review is endorsed by the Centers for Medicare and Medicaid Services which designates weekly physics and clinical management as a part of the treatment process. The American College of Radiology (ACR) accredits radiation oncology programs to demonstrate a minimum level of quality and safety. The radiation oncology standards set by the ACR require chart reviews to receive program accreditation.

Moreover, each radiation treatment produces hundreds of important data points. Subjects are typically treated with radiation every day for up to 40 treatments, producing many thousands of data points including images, delivery data from the treatment machine, and documentation. These data exist in multiple systems at multiple locations both in the data storage systems and in the user-facing systems to view the data. Access and review of this data can be a cumbersome and time-consuming task, which often limits the completeness of a chart review. In the modern age of computerization, the amount of data continues increasing at a rate that makes a complete review unrealistic. For example, the TPCR itself entails a multi-step verification of the defined treatment. It involves interaction with an inter-disciplinary team consisting of dosimetrists, radiation oncologists and therapists, and a detailed knowledge of multi-vendor software and hardware systems. The TPCR can involve the verification of more than 300 variables for completeness, integrity and consistency across multiple software applications and interaction with other staff members. However, a medical physicist is usually expected to perform the TPCR within 30 minutes to one hour and frequently has multiple charts to check simultaneously.

The current disclosure describes systems and methods for reducing error and increasing efficacy in performing QA and clinical workflow processes in radiation oncology. These systems and methods include interrogating EMR stored for radiation oncology subjects including, but not limited to, subject history, physician notes, diagnoses, prescriptions, physician orders, medical images, operative reports, test results, anatomic contours, treatment plans, other medical documents, and treatment-related information; aggregating; and analyzing the data. The system dynamically tracks the state of process variables in a subject's treatment chart and automatically identifies logical inconsistencies between variables, the severity of these inconsistencies, the context in which these inconsistencies arise, and how the inconsistencies propagate. This is designed to reduce the number of steps and time for verification of a subject's treatment chart at various stages of treatment, and to reduce error rates that exist in current manual processes.

It should be noted that unlike existing chart review and QA processes that analyze data using hard-coded rules and limits, the current systems and methods also propose a self-learning and classification component. Examples of the disclosed methods and systems can also improve themselves over time and repeatedly, for example using a framework for repeated training and learning. In some examples, the disclosed methods and systems may not be limited to a single site or technique but may generalize to multiple or all possible treatment sites and/or techniques, for example via the machine learning component with sufficient training data. This may be different from conventional approaches that are typically limited to single treatment sites and typically must be manually adapted to other sites, for example due to the lack of machine learning and/or generalized subject and plan features. Conventional methods are typically limited in the factors that are considered when performing QA or treatment planning. A classification and learning system may be able to incorporate other factors implicitly, without requiring a user to define specific parameters.

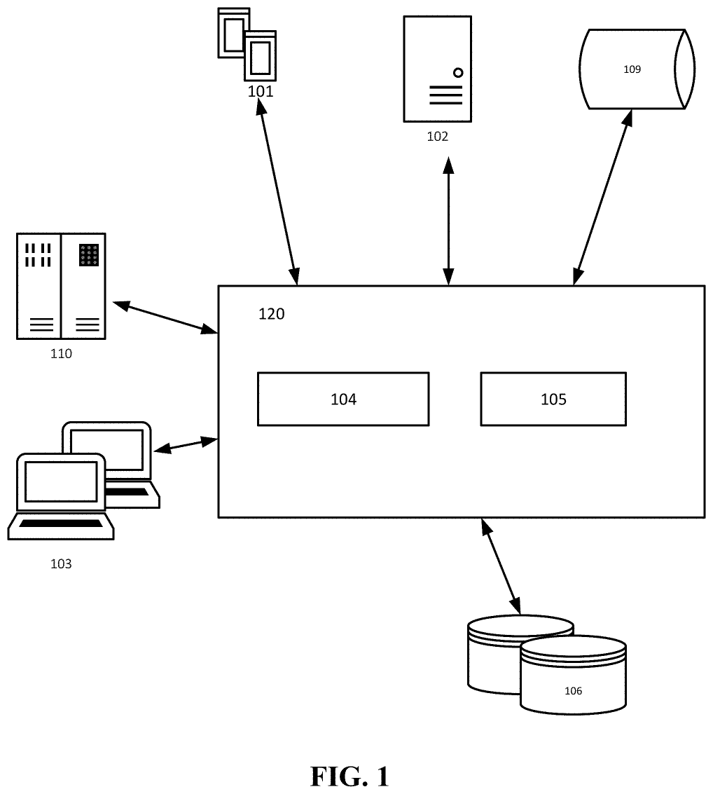

FIG. 1 illustrates an example system for providing automated quality assurance in radiation oncology. As shown in FIG. 1, the system may include image scanners 101 (e.g., magnetic resonance imaging (MRI), CT scanner, etc.), treatment planning system(s) 102, EMR system(s) 109, treatment delivery system(s) 110 (e.g., radiation therapy delivery machines such as linear accelerators, laser verification systems, etc.) and/or client device(s) 103 in addition to an automated QA and planning system 120, the example system may include only the automated QA and planning system, with the other components being separate from and external to the system. The example system may include a processor that communicates with one or more memories storing data and computer-readable instructions that, when executed by the processor, causes the system to carry out the disclosed methods.

The system may also include one or more data store(s) 106 that store one or more rules, training data, and/or historical data (e.g., plan features and subject features) and rules for implementing automated QA and/or automated treatment planning, as discussed below. The machine learning algorithms may be trained on a relatively large set of features. This database(s) 106 may be a storage location for all those features. The database(s) 106 may include a collection of high quality training data extracted from tens of thousands of clinical plans. The system may also communicate with one or more external data stores (not shown) for accessing such information. Information in the data store(s) may be continually updated (e.g., as new subject data and new treatment plans are available).

The example of FIG. 1 may implement a web-based module and visualization engine. The modular design of the system may limit the scope of each module to a specific task. This may allow a module to be reused in other systems. For example, the Artificial Intelligence (AI) module 105 may be used by a web application 104 for plan quality review and/or for automated treatment planning. The same module can also be used by a treatment planning system (discussed further below) to aid in identifying various technical parameters, for example. Modular design of the example system may also allow it to scale to support a growing user base. Each piece can run on separate clusters of servers. Alternatively, to save on costs it can all run on the same server. This modular design may enable separate presentation of various views of the data—which may be running on a client 103—from the massive amount of plan related data—which may be stored on a separate server.

In some examples, the system may include a web application 104 for receiving user input and providing the output, where the output is web-accessible (via, for example, the client device 103). This web 104 may tie together various algorithms and technologies and may present them to the user via a web interface. The web application 104 may be an implementation of a workflow engine for a radiation treatment plan—e.g., data constituting a proposed treatment plan may be passed through a rigorous clinical approval process with artificial intelligence algorithms aiding each step of the process, and may require a human user to sign off. Through the web application 104, the user can see the automatic artificial intelligence algorithm quality assessment, visualize all the RT treatment plan elements including subject scans, delineated regions of interest, beams and computed dose, and finally mark the plan as approved or rejected for delivery, for example. The web application may be stand-alone and/or may link to existing QA or planning software suites. The web application 104 may allow ubiquitous access. Users can use desktops, laptops and tablets, or any other suitable computing device to access the service. Users may have the ability to access the application 104 from anywhere in the world (e.g., using an internet connection).

Various components of the system may communicate with each other using any now or hereafter known communication protocols and networks (not shown here) such as, without limitation, Intranet, Internet, Bluetooth, Ethernet, or the like. Optionally, communications traffic may be encrypted (e.g., via HTTPS) and users may be required to be authenticated, to ensure security and subject privacy.

The example system may include a render server digital imaging and communications in medicine (DICOM) RT visualization module (not shown here). The DICOM RT Visualization module may handle render requests for DICOM treatment plan data. Algorithms in this module may be able to render various scanned images, beams, regions of interest, and dose. The module may be also able to compute dose-volume histograms. The module may expose its functionality via a remote procedure that calls API. This may allow web-based thin clients (such as the web application 104 running in a web browser) to render DICOM RT data for the user and may let the user to interactively visualize the data. The module may also include and/or may be in communication with a repository for storing DICOM treatment plan data. A high performance, DICOM compliant storage system may need to be used here to handle the relatively large number (e.g., tens of thousands) of plans the system may be expected to handle.

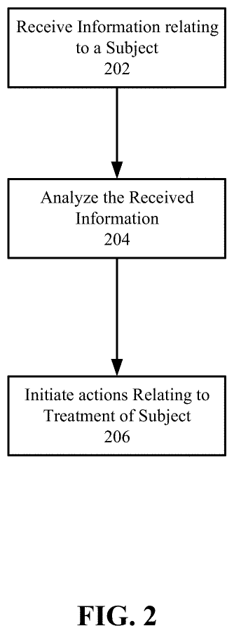

Referring now to FIG. 2, a flowchart illustrating an example method for performing automated quality assurance in radiation oncology is shown. The method can be performed in real or near-real time (e.g., as the image data is recorded, when treatment plan is being formulated, etc.), after a delay (e.g., using previously stored data), or at any suitable time. The method can be performed a predetermined number of times for a treatment plan, iteratively performed at a predetermined frequency for a treatment plan, or performed at any suitable time. Multiple instances of the method can be concurrently performed for multiple concurrent treatment plans (e.g., for different subjects, different treatment plans, etc.). However, any suitable number of method instances can be performed at any suitable time.

At 202, the system may receive information relating to a subject from, for example, various data store(s), imaging system(s), treatment plan system(s), treatment delivery system(s), and EMR(s). The system may receive the information based on, for example, subject identity. The received data may include, for example, a treatment plan(s) (that defined an RT treatment plan for at least one site), a subject characteristic, a subject history, a subject diagnosis, imaged feature(s), information relating to previously executed treatment plans or the like.

Optionally, the system may classify the treatment plan and other subject information according to one or more predefined features to determine the treatment plan classification. Classifying the proposed treatment plan may include determining a treatment plan class for the proposed treatment plan according to one of a plurality of predefined treatment plan classes. This may be carried out using an automated classification algorithm, as discussed below. In some examples, the automated classification algorithm may be developed by machine learning (e.g., based on Random Forest techniques, as discussed below) using historical data. Where machine learning is used, such learning may be ongoing, as additional historical data becomes available, such that the classification of a given treatment plan may be refined over time, for example. Classification of the proposed treatment plan, such as classifying the proposed treatment plan, may be based on determining the similarity (e.g., as calculated through distances) of features of the proposed treatment plan to features of known predefined treatment plan classes or characteristics. The proposed treatment plan may be classified according to various treatment plan features, for example, including one or more of: an anatomical site, a tumor histology, a prescription dose, a treatment technique, and a treatment intent. Optionally, treatment plans can be grouped by clinical technique such as isodose planning, depth calculation, 3D planning, intensity modulated radiation therapy, brachytherapy, proton therapy, electron therapy, etc. for a higher degree of accuracy.

Classifying the proposed treatment plan may also include determining a quality estimate that the proposed treatment plan belongs to for a given treatment plan classification, based on predefined expected features of the given treatment plan characterization. This may include identifying the clinical requirements and technical parameters of a treatment plan.

The system may then analyze (204) the received data based on one or more rule sets and/or machine learning models to determine whether the received information satisfies one or more subject treatment requirements, clinical requirements, safety standards, compliance standards, or the like. For example, the system may use the determined quality estimate, expected treatment plan features, and/or clinical requirements to determine whether there are any safety concerns, quality issues, compliance issues, or the like with respect to the treatment of a subject. The analyses may also include determination of how well certain plan features (e.g., technical parameters) match with expected plan characteristics (e.g., clinical requirements) and may include generation of error and/or warnings if the quality estimate is below a certain threshold value, compliance or safety issues are identified, or the like. For example, the quality estimate may be calculated as a confidence value, a simple pass/fail determination, or any other suitable estimate of quality.

The analyses may include evaluating the information about a subject (e.g., a treatment plan) according to one or more rules defining expected relationships between the clinical requirements of a treatment plan and one or more of: one or more plan features, technical parameters, and one or more subject features defined in the set of subject data. The rule(s) may be defined (e.g., predefined manually) or machine-learned based on historical data of historical treatment plans, as discussed further below. Where machine learning is used, such learning may be ongoing, as additional training data becomes available, such that the rule(s) may be refined over time, for example. The training data may include historical data of historical treatment plans such as treatment outcome data. Rules defining expected relationships may include, for example, one or more of: historical suitability of a given treatment plan characterization for historical subjects; historical treatment outcome of a given treatment plan characterization for historical subjects; historical treatment plans for a specific subject; historical treatment outcomes for the specific subject; a mathematical function; and a general rule governing treatment plans irrespective of the treatment plan characterization and irrespective of the subject data; among others.

Examples of some of these analyses are provided below:

For example, in certain implementations, the system may analyze information relating to the technical execution and/or technical parameters of a treatment plan to determine whether or not the technical plan meets various safety and/or compliance standards. The system may analyze data such as, for example, parameters of technical plan delivery system(s) (e.g., accelerometers), time and monitor units of execution, x-ray images acquired, respirator gating data, surface alignment & monitoring data, and other system parameters recorded during treatment (such as component and fluid temperatures, voltages, currents, and timing). Treatment delivery technical information includes, without limitation, treatment couch coordinates, gantry coordinates, collimator coordinates, multileaf-collimator positions, monitor units, dose rate, energy, faults, and interlocks for each treatment field (treatments typically consist of 2 to 6 fields), or the like. Examples of the treatment delivery parameters can include raw treatment delivery execution results including mechanical movements of the linear accelerator gantry components (e.g., collimator and gantry angle), couch (e.g., pitch, yaw, roll, x/y/z-axis translations), imaging systems (arms of the imaging source and detector, detector translation position, filter position), beam line components (jaws, micro-leaf collimators, secondary jaws, carriages, filters, targets/windows, microelectronic modes). The system may then analyze the parameters by comparing them against standard ranges for each parameter. Alternatively and/or additionally, trends relating to a parameter may be determined using statistical process control to monitor changes that are noteworthy when compared as a set. Noteworthy data may be automatically determined using, for example, statistical process controls defined (e.g., Western Electric rules) and/or a multi-layer machine learning model with nodes for each input as well as hidden abstraction and convolution layers to identify anomalies.

The system may, therefore, output anomalies that require special investigation by the user of the system that might not otherwise have been detected by a simple data review or infeasible for a human to complete in a timely fashion due to the large volume, complexity, and/or interdependence of the parameters that cannot be observed by a human or even a statistical trend analysis.

In various other implementations, the system may analyze the technical parameters of a treatment plan execution to determine whether or not the desired treatment was delivered (i.e., the clinical requirements were met). Similar to the previous example, the treatment plan delivery system executes technical parameters from the treatment planning system to meet a subject's treatment objectives. In this implementation, the analysis is performed as above with the desired treatment plan configuration (i.e., the clinical requirements) as an additional input. Differences between the desired state and the executed state may be evaluated in the same manner as above. Other factors such as the standard range of acceptable deviation, learned range based on the performance of the particular equipment and/or other unspecified but learned factors may be taken into account.

Again, the recorded parameters are too numerous for a human (and/or simple trend analysis) to identify complex inter-related patterns that are only identified through machine learning methods of this disclosure that learn from training data including historical data. The system may also be able to analyze parameters from multiple subjects and/or across multiple systems for inter-machine and/or inter-subject variable comparison that would not be obvious to a human user. For example, the system may use a machine learning algorithm to analyze the trend of technical parameters both from an individual subject and the aggregate of all subjects to determine if an anomaly has occurred.

As an illustrative example, a treatment scenario is considered in which a subject is setup for radiation treatment in a certain couch position on the first day and images are acquired, subsequent to which manual review may conclude that the setup is accurate and elect to proceed with treatment. The next day the subject returns and is setup on the couch where the positions indicate a vertical deviation from the previous value that is not flagged by a rules-based algorithm as configured by the administrator of the system (because, for example, it falls within the allowed tolerances). However, the result is outside of normal limits of variation identified by the system using machine learning and flagged for review. An alert may then be generated immediately and automatically (discussed below). Similarly, in the above example if on the next day the subject setup requires a shift that is within the tolerance established by the rules, and/or the trend is not yet long enough to establish a variation history. However, the system can flag this change when other subjects with the same diagnosis and treatment delivery systems typically fall within much tighter couch position differences than observed for this treatment. In another example, a treatment scenario is considered where a subject requires a bolus to be added and the bolus information to be stored in the planning system. That bolus is also recorded to be used by a clinical device that scans the bolus each time it is placed for treatment. If the treatment occurs without bolus and may not caught by either the clinical staff or the treatment system. However, the present system may recognize that bolus should be scanned for plans that also contain bolus specifications, and if a scan is absent the system may take an action such as alert a user and/or prevent the treatment from going forward. The resulting action is again to create an alert to users. Additionally and/or alternatively, that bolus may be commonly used for this particular disease indication, and if forgotten completely, the system can flag the anomaly based on other subjects bolus use.

In additional to the treatment plan execution, the treatment plan itself can be analyzed to interpret technical parameters of a treatment plan and/or identify anomalous parameters by, for example, comparing other plans corresponding to the same or different subjects to determine if the plan contains unusual features and/or errors.

In some implementations, the system may perform the analyses to determine whether the required documentation exists in various EMR(s) for a subject before, during, and after a treatment plan delivery. Documents are required for each step of a clinical process. The components and language of each document have specific guidelines or requirements. The system may compare the existing documents associated with a subject with other documents in the EMR (e.g., documents of other subjects with similar treatment plans and/or diseases) to identify discrepancies, errors, and/or abnormalities. The system may, for example, use any now or hereafter known natural language processing techniques or other machine learning models for performing document analysis. For example, the system may identify key words and phrases, and use them as inputs into a machine learning mode (e.g., a neural network, a deep learning model, etc.) to identify missing or abnormal key words/phrases. These may be reported to the user to alert them to abnormalities that need further validation, investigation, and correction. If the user finds them acceptable, the new data may be used as feedback to train the neural network either periodically or after each review by a user.

Optionally, the system may also compare the language and/or content of document(s) to radiation therapy-specific data points (such as the clinical documentation, treatment plan, treatment delivery documentation, chart review and analysis, and other relative parameters) for determining whether certain subject specific documents are missing (i.e., verifying all necessary documentation exists), include errors, can be improved, or the like. For example, the system may compare the documents to discrete data points from a treatment plan (e.g., any element including the prescription, normalization points, treatment volumes, organs at risk, technique, treatment fields, imaging fields, digitally reconstructed radiographs (DRRs), mode, energy, calculation algorithms, etc.); data points from other types of clinical documentation (notes, phone records, flowsheet text, etc.), the parameters previously discussed regarding treatment delivery, and trend/analysis outputs, etc. to determine if additional documentation should be used for a subject.

Similarly, the system may analyze medical images using various now or hereafter known image processing methods and algorithms to verify the quality of acquired images, conformance with treatment plan requirements and/or to automatically identify changes in the images associated with a subject. Examples of such algorithms may include, for example, mutual information algorithms, neural networks, deep learning models (e.g., deep learning convolutional neural networks or DLCNN), and matrix-based pixel comparisons. In DLCNN, the system uses multiple hidden layers beyond the initial input layer multiple hidden layers beyond the initial input layer. The voxels from both images in the comparison are provided to the input layer, and then the hidden DLCNN layers determine the statistical similarity of the two images. Optionally, the system may receive outcome data in the form of physician suggested positional shifts and structured reports which may be used to continually refine the model. In various embodiments, the system may perform an image analysis each time a medical image of the same type and position (as previously acquired for the subject) are acquired during the treatment process.

In certain implementations, the system may perform the analyses to verify whether orders and prescriptions are accurately fulfilled by clinical actions including treatment plans, dose distributions, dose volume statistics, medical imaging, and medical treatments. For example, the system may verify that the radiation dose and prescription information entered by a physician is correct and consistent across all components of the system, as well as accurate and proper administration.

As discussed, treatment plans, orders, prescriptions, medical images, documents and treatment data may be stored in different systems, formats, and data stores. The system may be configured to access the data in the native format of each system through an application programming interface (API) (such as FHIR (fast healthcare interoperability resource), Health level 7 (HL7), SQL, DICOM) and/or proprietary formats. The system may then map the data between ordered and prescribed elements (the inputs) and the outcomes (the outputs). Outcomes may include treatment plan parameters (e.g., technique, mode, energy, etc.), type/quantity/age of medical images, supporting documentation, resulting treatment plan dose distributions, dose volume statistics, medical treatment data, or the like. In addition to displaying this data, the system may also automatically verify that each mapped relationship is fulfilled—i.e., the orders and prescriptions are accurately fulfilled (instead of manual verification). Each requested order is fulfilled by a process that generates discrete electronic data (e.g. a request for a CT scan of the head should produce a set of CT images in DICOM format) or free form electronic data (e.g. a request for physics case review would generate an unstructured report). The order can be matched to the output through a combination of rules-based comparisons and/or machine learning tools. In the examples above, a simple rules based comparison may show that a CT image was acquired after the order was created, while an machine learning analysis of the image set may show that it has captured the head region. This may be verified at each clinical step and/or the verification may occur upon detection of EMR task status changes, treatment plan status changes, prescription status changes, the presence of new medical images within DICOM repositories or databases, and/or the presence of new treatment data. Optionally, these verifications may be performed in real time.

In one or more implementations, the system may also perform the analyses to determine whether a subject needs a chart review. A chart review evaluates the correctness of one or more variable or parameters of a treatment by evaluating them to see if they violate one or more clinical requirements; and may be performed periodically and/or upon occurrence of a particular event. For example, if the system identifies discrepancies in treatment plans, orders, prescriptions, execution of treatment plans, etc. the system may determine that the subject requires a chart review. In another example, a chart review may be initiated if certain types of new data (e.g., images, prescriptions, etc.) are added to the system. Optionally, the system may perform a rules-based analysis that can be configured by an administrator to meeting billing or regulatory requirements relating to manual chart reviews. The system may notify a user that a chart review is required and/or may perform the chart review automatically. For example, an automatic chart review process may analyze all data and learned insights related to a course of medical treatment, which includes, without limitation, patient history, physician notes, diagnoses, prescriptions, physician orders, medical images, operative reports, test results, anatomic contours, treatment plans, other medical documents, and treatment-related information (e.g., technical parameters, treatment delivery technical information, etc.) to verify their compliance with clinical requirements.

Chart review in radiation oncology occurs at frequencies prescribed by accredited medical organizations, such as the ACR, and insurance payers. A typical frequency is one check pre-treatment, then an additional check once every five treatments. To satisfy safety and payment requirements time and effort must be spent to ensure these checks occur at the appropriate times. It is not uncommon for a patient to miss an appointment or for equipment malfunctions to cause treatment delays. The current system may automatically tracks the recommended or necessary time for chart review based on treatment plan status and delivered treatments. The system may then provide records to the end user at the correct time for review and may alert the end user if certain cases are close to becoming overdue for review.

Other analyses may include, without limitation, identifying issues within a chart, identifying issues relating to a chart review process, tracking changes to a chart over time, facilitating a checklist of items that have been or should be completed as a part of the chart review, learning new parameters that may require additional consideration as a part of the chart review and analysis process, or the like.

In certain examples, when new data is added to any of the peripheral modules/systems connected to the present system, the system may identify the formatting and use of such data. For example, if a particular system communicates a JSON object including critical treatment plan parameters, new or novel parameters may be discovered by comparing data from several points in time in the past from the same system, and then identifying differences. These differences may be stored for an administrator to include in the algorithm or rules-based functionality. In the case that the system is using machine learning, additional nodes may be added at the first layer of the ML input to accommodate the new data, at least one per discovered item. The inclusion of the new data may trigger a retraining of the model which is then used in the analysis process moving forward.

Based on the analysis, the system may initiate one or more actions relating to the treatment of the subject (e.g., chart review process) (206). These actions can be performed in real time. For example, if a change to a treatment plan is made that has inconsistencies or abnormalities, the system may immediately prevent treatment plan execution by changing the approval status of the treatment plan preventing delivery errors.

Examples of such actions may include without limitation, providing an alert, configuring a user interface to appropriately display results of the analyses, initiate an action to prevent further treatment related actions until a flagged issue has been resolved, storing results of the analyses to a data store, updating a machine learning model, initiating a chart review, creation and/or auto-filling of one or more documents, performing a corrective, or combinations thereof. These actions are described below in detail. It should be noted that the above analyses also include identifying various corrective actions that a user and/or system can undertake in response to identifying discrepancies, errors, missing documents, QA issues, compliance issues, safety issues, or the like. The system may, therefore, prompt a user to perform the identified corrective action and/or perform the actions automatically. For example, if an error is identified in an imaging order, the system may automatically generate a new imaging order that complies with the clinical requirements.

In certain implementations, the system may generate different types of alerts in response to identifying (in step 204), for example, discrepancies, deviations, errors, trends, or the like, in order to assist a clinical staff person(s) in optimizing a subject's treatment outcome. The system may include one or more triggers that may lead to issuance of an alert to cause a clinical person (s) to review certain information and/or alert them to them to abnormalities that need further validation, investigation, and/or correction. Examples of such alerts may include, without limitation visual alerts on a client device (e.g., flags, color coded alerts, text alerts, messages, emails, pop-ups, etc.), sound alerts (e.g., alarms, broadcasts), haptic alerts, or combinations thereof. The alerts may include, for example, best practice advisories, warnings, flagged actions, next steps, corrective actions, or the like. Optionally, a user may interact with the alert (e.g., click on the alert) to receive more information about the alert. In various examples, the alerts may be interfaced by system through multiple forms of APIs including FHIR, HL7, web hooks, and custom formats. The system may, optionally identify users who should receive the alerts and/or their alert format preferences.

For example, the system may be configured to either automatically or on-demand audit an entire subject record (e.g., upon completion of a treatment plan) to determine if all the necessary documentation has been generated and is correctly associated with corresponding data such as orders, prescriptions, or treatment plans. Users may be informed if the system identifies incomplete documentation, medical information, technical parameters, or other information needed for current or future care or administrative uses such as subject billing or statistics.

In certain other implementations, the system may also configure a user interface (e.g., graphical user interface) to present various information, charts generated based on the above analyses, data, and/or reports of the analyses. Such user interface may be displayed on a client device using, for example, a graphical user interface. The user interface may be configured to display, for example, raw data, trends, statistical parameters, rolling data windows (i.e., rolling windows over several data points), identified anomalies, images, documents, notes, and interpreted parameters and analyses, or the like. The system may also create new visualizations of chart and medical data insights that a user may interact with (e.g., select, manipulate, delete, move, etc.). For example, a user may choose a data point (such as an identified anomaly) to cause a displayed graph to change to show similar data with presence or lack of anomalies presented. This may lead to new insights about a broader trend that may be understood by a clinical user but not apparent (from the machine learning analysis itself) unless the relevant data is appropriately discretized for presentation to a user. Optionally, the system may configure the user interface to present the most important data first (e.g., warnings, alerts, corrective actions that require immediate user action) before a user can view other information or data.

The system is capable of identifying changed parameters for display to the end user using the user interface of this disclosure. Changes to parameters of an order, prescription, plan, or treatment may be displayed in linear chronology over time. Important changes may be highlighted or flagged in a manner that require review. If a change presents a new hazard that should be corrected prior to the patient continuing in the clinical process, the system can automatically disable or remove approval of the treatment plan to prevent treatment and notify the correct users.

The system provides a customized user interface to view various subject charts, i.e. data aggregated and analyzed by the system for facilitating rapid access to medical information that includes key insights (e.g., trends, discrepancies, QA information, etc.). The data may be presented in a condensed format with data layers cascading in a hierarchy. The highest layers of hierarchy may be presented to the end user along with any warnings that are contained within that cascade streamline. If the user elects to perform further analysis or exploration, the next layer of the hierarchy may be accessed and displayed, again showing streamline warnings. Thus, the system quickly and efficiently leads users to specific data identified to have anomalies or clear rule violations based on the analysis previously performed. For data where automated solutions are available, the user may click a button to fix the data. In this case, the system may interface with the data endpoint (e.g., EMR, treatment plan, etc.) and fix the data based on a suggested value from the rules and/or analysis. For example, a treatment plan may contain a treatment field with the wrong naming format that is flagged by the system. The correct naming format may be identified based on rules and/or learning models (and may, optionally, be provided to a user for approval/review). The system may access the treatment planning system, open the existing treatment plan, disable it from clinical use temporarily, change the field name to the correct value, reapprove the treatment plan for clinical use, and close the treatment plan. The completion of this task may reported back to an appropriate user.

The interface may also be configured to allow manipulation of the data to show graphical relationships between data layers that have been used to develop the insights described above. The system can, therefore, offer end users the choice to explore cause and effect relationships in the data. In an embodiment, the users' experience may be graphical, visualizing large datasets with an approachable and navigable interface, allowing users to pose “what-if” questions in real-time, and analyze potential outcomes, and select appropriate pathways. The data visualization can allow drilling into the graphic datasets, and re-arranging metrics to ask questions in different ways. Potential outcomes can then be saved as an individual user's personal choice profile, for later reference and further exploration of the information. In an example, the system may utilize vast amounts of data using machine learning algorithms to interpret real-time changes and thereby provide various treatment adaptations in real-time.

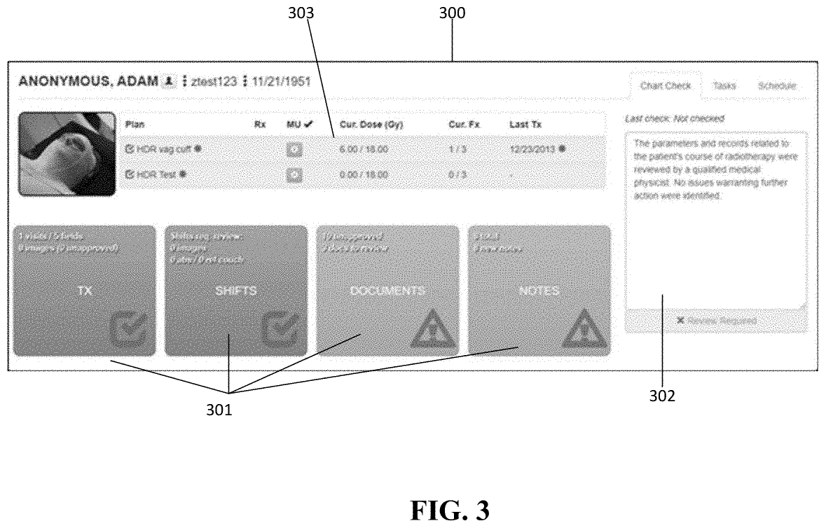

Optionally, the system acquire all data that is needed for a chart review, process the data into actionable items, and present the data to the end user in a custom designed badge display such the example user interface 300 shown in FIG. 3. In FIG. 3, one or more sections (e.g. treatment, imaging/positioning, medical documents, staff notes, and treatment plans/prescriptions/orders/secondary checks sections) are represented as badges 301. Badges and all data comparisons may be shown with contextual colors that indicate, for example, green if there are no warnings detected, orange if a review is required, red if the treatment has been stopped, or the like. The system may further indicate warnings section 302 since the most recent chart review to aid in solving relevant and/or un-reviewed problems. If any new warnings are discovered, the badge may be capable of changing color to orange, yellow, or red, appropriate based on context, to alert the end user of an issue. Issue information including, but not limited to, a summary of problems, summations of multiple errors or warnings, or clinical advisories may also be listed on the badge 301. Optionally, such information may be accessed by clicking on one or more tabs within a badge. Any badge may be expanded to show data relevant to the warnings. The badge may allow for further selection to display all data pertaining to the current area of interest and/or warnings that the badge represents. Optionally, the system may not allow completion of the chart review until badges with warnings have been expanded and reviewed and/or acknowledged. Any required sub-section reviews, such as document review as previously described, may keep the badge in a warning state until complete. Current tests and prescriptions may also be shown (303). As discussed, the information may be sorted and displayed in a hierarchical manner such that the most important information may be displayed to the user first followed by other information (e.g., after receipt of a user interactive action).

The treatment section acquires all the expected and actual values for the various parameters of radiation treatment including, but not limited to, table, gantry, collimator, jaws, overrides, interlocks, monitor units, dose rates, plan status, and prescription status. Any deviations outside a specified tolerance, issues that have been flagged by the automated analysis including methods such as statistical process control, or parameters that experienced a change in status creates a warning and presents the relevant data for review. Values without issue can be automatically suppressed and are directly available on request.

The imaging/positioning section acquires table positions for imaging and treatment, the number and type of images, the shifts from imaging to treatment, and other quantitative data related to treatment alignment or accuracy. Any shift of a parameter such as a table position that falls outside of a defined tolerance will create a warning and present the relevant data for review. The mean of the table positions and shift values are tracked over all treatments, and any single value that falls outside a defined number of standard deviations from the mean will trigger a warning.

The medical documents section presents all relevant documents from the various EMR systems. Documents of predefined types that have been created since the most recent chart check are presented for review. All other documents are directly available at request. Documents types can be assigned to specific roles for required review and filtered based on time periods or automatically suppressed if created prior to the last chart check. Important documents can be flagged for review by groups of users based on other user activity or phrases/values that may indicate that special attention is needed. The system can be configured to block completion of the chart check until all required documents have been reviewed by the end user.

The staff notes section presents all relevant notes from the various EMR systems aggregated into one place. Notes that have been created since the most recent check by the specific staff role are presented for review. All other notes are directly available at request. The notes can be filtered by date and/or type.

The system may also be configured to track changes to a subject's chart (or other information) over time and present it to a user in an easy to interpret visualization using the user interface. For example, as chart data evolves during a subject's treatment, the system may identify changes to discrete values in the disparate systems and show a summary of those changes to the end user. Specific change data may also be available for visualization by selection of a specific data element that a user wants to investigate (e.g., upon selection of a particular data point, specific information relating to that data point may be displayed within the same interface). For numeric or discrete data, the user can interact with the data in real-time, exploring trends, charts, etc.

In some example embodiments, the system may also review information relating to various user access levels (or permissions) and configure the user interface based on such user access levels. For example, if a user does not have access to EMR data and/or treatment plan of a patient, but can perform a corrective action, the system may configure the user interface to only present the allowed information to the user and disable access to any other information.

In some implementations, the system may automatically disable (e.g., using appropriate APIs) one or more components associated with a treatment plan as a form of action in response to the analyses of the above step. For example, upon detection of an anomaly in a treatment plan, the system may disable the treatment planning system and/or the treatment delivery system from moving a subject forward in the clinical process (or being used in treatment) until, for example, after the anomaly is resolved and/or after manual intervention. For example, if a change to a subject's treatment plan is made that is determined to have inconsistencies or abnormalities the system may immediately prevent further treatment by changing the approval status of the care plan preventing treatment delivery errors.

In some other implementation, the system may store various results of the analyses in a data store (e.g., an incidence reporting system, EMRs, oncology information systems, training data stores, etc.) for future use, verification, correction, or the like. For example, the results may be used for training and/or updating the machine learning models described in this disclosure. For example, a user interface during a chart review system may include buttons that enables quick reporting of any anomalies or patient safety events identified by the user at the time of the chart review. The system may generate a report(s) that captures and transmits the user commentary (if provided) along with the subject data including all or some of the received information and the results of any analysis of the system. The system can maintain a list of these reports within data stores and display them in an aggregate list. Data inside these systems are stored in multiple views and pages.

In certain implementations, the actions may include intelligently creating document templates, presenting the documents to a user in an interactive interface to receive information for filling the document, and/or auto-filing (partially and/or completely) documents (e.g., when documents are determined to be absent, including incorrect information, or the like). For example, the system may create a document template for a treatment plan based on documents previously created for similar treatment plans. Examples of such documents may include, without limitation, special physics consult reports, physician treatment visit summaries, treatment plan summary notes, special treatment procedure notes, treatment visit notes, or the like.

The system may also facilitate document creation relating to a chart check or review (i.e., during and/or after a user is reviewing a chart). The system may build a document relating to a chart check continuously based on the specific actions of a user performing the chart check. The actions include review of medical records, images, trends, analysis data, alerts, and errors. As these items are accessed or analyzed during the chart review, the system builds the narrative in a format that can be easily viewed by a user. The narratives can be adjusted or improved by the user manually based on the review. Once the review is complete, the documentation and narrative of the chart check may be assembled into a durable document format, and sent to the appropriate EMR location. This is configurable to include multiple EMR systems or other systems capable of storing medical documentation. The system can create a document to record the chart check in the EMR. The document can be automatically formed and populated with any data point(s) reviewed during the chart check. It also can be populated with a quick narrative using pre-filled default values. The system, optionally, may not allow automatic creation of the document until the chart check is considered complete.

In certain examples, the system may display a series of checklist items that are either required or optional/suggested during a chart review by a user. The checklist can be manually completed by the user by checking boxes next to items, or the checklist can be automatically completed either as the user reviews specific pieces of information/alerts or after the system completes its automatic analysis of items previously mentioned. The checklist may be stored in the system's database and can be recalled and/or included in the chart review documentation. Warnings or alerts for individual items can cause specific items in the checklist to appear different or have additional text pointed the user to an element that has been identified by the system.

The system may, optionally, identify the clinical staff person(s) who should be related to the actions. For example, the system may identify which personnel should view and complete various required actions (e.g., chart reviews) using a rule-set and/or a machine learning model that adjusts to labor utilization such as staff being consumed by other tasks or having capacity for additional effort. Users can be given charts to review based on current workload, number of previously reviewed charts within defined time periods, or status within the EMR system (i.e. on-site versus on vacation). The system may also track various users currently reviewing a chart. For example, the system may tag a chart with a user's identity upon commencement of a review. The data and time may be stored along with the user information. This data may be displayed to other users to prevent overlapping chart review. During a chart check, the system may indicate to all users that a specific user has started the check. This avoids duplicate work when multiple end users with the same role or responsibilities are working from the same web-based worklist.

Chart review in radiation oncology occurs at frequencies prescribed by accredited medical organizations, such as the ACR, and insurance payers. A typical frequency is one check pre-treatment, then an additional check once every five treatments. To satisfy safety and payment requirements time and effort must be spent to ensure these checks occur at the appropriate times. Systems and methods of the current disclosure automatically track the recommended or necessary time for chart review based on treatment plan status and delivered treatments. The system may only provide records to the end user at the correct time for review and alerts the end user if certain cases are close to becoming overdue for review.

Although described with respect to cancer conditions, the described clinical outcome therapeutic analysis can be used for any clinical condition (e.g., cardiovascular disease, metabolic disease (diabetes), immune mediated diseases (e.g., lupus, rheumatoid arthritis), organ transplantation; neurodegenerative disorders; pulmonary diseases, infectious diseases, hepatic disorders, or the like.

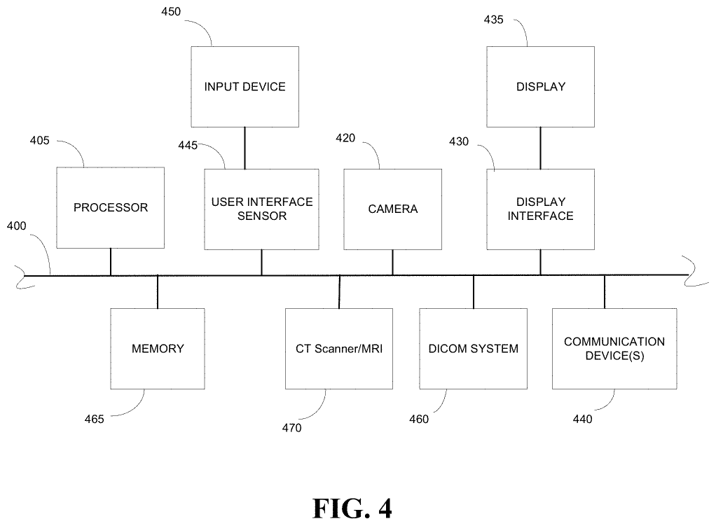

FIG. 4 depicts an example of internal hardware that may be included in any of the electronic components of the system, such as the QA system, the treatment planning system, or remote servers. An electrical bus 400 serves as an information highway interconnecting the other illustrated components of the hardware. Processor 405 is a central processing device of the system, configured to perform calculations and logic operations required to execute programming instructions. As used in this document and in the claims, the terms “processor” and “processing device” may refer to a single processor or any number of processors in a set of processors that collectively perform a set of operations, such as a central processing unit (CPU), a graphics processing unit (GPU), a remote server, or a combination of these. Read only memory (ROM), random access memory (RAM), flash memory, hard drives and other devices capable of storing electronic data constitute examples of memory devices 425. A memory device may include a single device or a collection of devices across which data and/or instructions are stored. Various embodiments of the invention may include a computer-readable medium containing programming instructions that are configured to cause one or more processors, print devices and/or scanning devices to perform the functions described in the context of the previous figures.

An optional display interface 430 may permit information from the bus 400 to be displayed on a display device 435 in visual, graphic or alphanumeric format, such on an in-dashboard display system of the vehicle. An audio interface and audio output (such as a speaker) also may be provided. Communication with external devices may occur using various communication devices 440 such as a wireless antenna, a radio frequency identification (RFID) tag and/or short-range or near-field communication transceiver, each of which may optionally communicatively connect with other components of the device via one or more communication system. The communication device(s) 440 may be configured to be communicatively connected to a communications network, such as the Internet, a local area network or a cellular telephone data network.

The hardware may also include a user interface sensor 445 that allows for receipt of data from input devices 450 such as a keyboard or keypad, a joystick, a touchscreen, a touch pad, a remote control, a pointing device and/or microphone. Digital image frames also may be received from a camera 420 that can capture video and/or still images. The system also may receive data from a image scanning device such as a CT scan/MRI machine 470 such as an accelerometer, gyroscope or inertial measurement unit. The system also may receive data from a DICOM system 460 such as that described earlier in this document.

The above-disclosed features and functions, as well as alternatives, may be combined into many other different systems or applications. Various components may be implemented in hardware or software or embedded software. Various presently unforeseen or unanticipated alternatives, modifications, variations or improvements may be made by those skilled in the art, each of which is also intended to be encompassed by the disclosed embodiments.

1. An oncology radiation therapy treatment review system, the system comprising:
a radiation therapy delivery system;
a processor; and
a non-transitory memory containing computer-readable instructions that when executed will cause the processor to:
receive information relating to a subject undergoing radiation therapy treatment in accordance with a treatment plan,
identify at least one clinical requirement associated with the treatment plan, and
analyze the received information and the at least one clinical requirement for identifying an action, the action comprising at least one of the following: configuring a user interface, performing a corrective action, storing results of the analyzing to a database, outputting an alert, or disabling treatment, or a document related action.

2. The oncology radiation therapy treatment review system of claim 1, further comprising computer-readable instructions that when executed cause the processor to control the radiation therapy delivery system to deliver the radiation therapy treatment.

3. The oncology radiation therapy treatment review system of claim 1, further comprising computer-readable instructions that when executed cause the processor to analyze a plurality of technical parameters of the treatment plan and using the analysis to control the radiation therapy delivery system to deliver the radiation therapy treatment.

4. The oncology radiation therapy treatment review system of claim 3, wherein the plurality of technical parameters comprise at least one of: gantry angle, collimator angle, beam isocenter position, collimator jaw setting, beam placement, beam shaping, dose distributions, dose volume statistics, or beam strength.

5. The oncology radiation therapy treatment review system of claim 3, wherein the at least one clinical requirement comprises at least one of the following: amount of non-target tissue that may be irradiated, minimum amount of coverage of the target area, prioritization of areas to irradiate, or prioritization of areas to avoid.

6. The oncology radiation therapy treatment review system of claim 1, further comprising a display coupled to the processor for displaying the user interface, and an input device coupled to the processor for receiving a user input.

7. The oncology radiation therapy treatment review system of claim 6, wherein the action comprises configuring the user interface to display the received information and results of the analyzing in a hierarchical manner.

8. The oncology radiation therapy treatment review system of 1, wherein the instructions that when executed cause the processor to analyze the received information and the at least one clinical requirement for identifying an action further comprise instructions that cause the processor to:
identify one or more errors in documentation required for the treatment plan;
identify a corrective action corresponding to the one or more errors; and
perform the corrective action.

9. The oncology radiation therapy treatment review system of 1, wherein the instructions that when executed cause the processor to analyze the received information and the at least one clinical requirement for identifying the action further comprise instructions that cause the processor to disable the treatment delivery system until one or more discrepancies identified in the treatment plan have been resolved by the system.

10. The oncology radiation therapy treatment review system of 1, wherein the action further comprises at least one of the following: initiating a manual chart review or performing an automatic chart review.

11. The oncology radiation therapy treatment review system of 1, wherein the instructions that when executed cause the processor to analyze the received information and the at least one clinical requirement for identifying the action further comprise instructions that cause the processor to automatically identify one or more errors in a prescription or an order associated with the treatment plan.

12. The oncology radiation therapy treatment review system of 1, further comprising instructions that when executed cause the processor to automatically identify the treatment plan.

13. The oncology radiation therapy treatment review system of 1, wherein the instructions that when executed cause the processor to analyze the received information and the at least one clinical requirement for identifying the action further comprise instructions that cause the processor to determine compliance with one or more quality assurance rules relating to the treatment plan.

14. The oncology radiation therapy treatment review system of 1, wherein the instructions that when executed cause the processor to analyze the received information and the at least one clinical requirement for identifying the action further comprise instructions that cause the processor to determine one or more errors associated with a plurality of images corresponding to the treatment plan.

15. An oncology radiation therapy treatment review method, the method comprising, by a processor:
receiving information relating to a subject undergoing radiation therapy treatment in accordance with a treatment plan;
identifying at least one clinical requirement associated with the treatment plan; and
analyzing the received information and the at least one clinical requirement for identifying an action, the action comprising at least one of the following: configuring a user interface, performing a corrective action, storing results of the analyzing to a database, outputting an alert, or disabling treatment, or a document related action.

16. The oncology radiation therapy treatment review method of claim 15, further comprising controlling the radiation therapy delivery method to deliver the radiation therapy treatment.

17. The oncology radiation therapy treatment review method of claim 15, further comprising analyzing a plurality of technical parameters of the treatment plan and using the analysis to control the radiation therapy delivery method to deliver the radiation therapy treatment.

18. The oncology radiation therapy treatment review method of claim 17, wherein the plurality of technical parameters comprise at least one of: gantry angle, collimator angle, beam isocenter position, collimator jaw setting, beam placement, beam shaping, dose distributions, dose volume statistics, or beam strength.

19. The oncology radiation therapy treatment review method of claim 17, wherein the at least one clinical requirement comprises at least one of the following: amount of non-target tissue that may be irradiated, minimum amount of coverage of the target area, prioritization of areas to irradiate, or prioritization of areas to avoid.

20. The oncology radiation therapy treatment review method of claim 19, further comprising displaying the user interface on a display device.

21. The oncology radiation therapy treatment review method of claim 20, wherein the action comprises configuring the user interface to display the received information and results of the analyzing in a hierarchical manner.

22. The oncology radiation therapy treatment review method of 15, wherein analyzing the received information and the at least one clinical requirement for identifying the action further comprises:
identifying one or more errors in documentation required for the treatment plan;
identifying a corrective action corresponding to the one or more errors; and
performing the corrective action.

23. The oncology radiation therapy treatment review method of 15, wherein analyzing the received information and the at least one clinical requirement for identifying the action further comprises disabling the treatment delivery method until one or more discrepancies identified in the treatment plan have been resolved by the method.

24. The oncology radiation therapy treatment review method of 15, wherein the action further comprises at least one of the following: initiating a manual chart review or performing an automatic chart review.

25. The oncology radiation therapy treatment review method of 15, wherein analyzing the received information and the at least one clinical requirement for identifying the action further comprises automatically identifying one or more errors in a prescription or an order associated with the treatment plan.

26. The oncology radiation therapy treatment review method of 15, further comprising automatically identifying the treatment plan.

27. The oncology radiation therapy treatment review method of 15, wherein analyzing the received information and the at least one clinical requirement for identifying the action further comprises determining compliance with one or more quality assurance rules relating to the treatment plan.

28. The oncology radiation therapy treatment review method of 15, wherein analyzing the received information and the at least one clinical requirement for identifying the action further comprises determining one or more errors associated with a plurality of images corresponding to the treatment plan.
